(19) United States
(12) Patent Application Publication
(10) Pub. No.: US 2022/0338802 A1
(43) Pub. Date: Oct. 27, 2022

(54) ORTHODONTAL CORRECTION MONITORING AND COMMUNICATION SYSTEM AND METHOD OF USE

(71) Applicant: In Hand Health, LLC, Overland Park, KS (US)

(72) Inventors: Michael J. Gassman, Kansas City, MO (US); Jeffrey S. Clary, Overland Park, KS (US); Stephanie M. Nicholson, Leawood, KS (US); Christopher W. Maloney, Overland Park, KS (US); Kurt Hoffman, Leawood, KS (US)

(21) Appl. No.: 17/862,599

(22) Filed: Jul. 12, 2022

Related U.S. Application Data
(63) Continuation-in-part of application No. 16/594,558, filed on Oct. 7, 2019, now Pat. No. 11,387,006.
Application No. 16/594,558 is a continuation of application No. 17/095,933, filed on Nov. 12, 2020, now abandoned.
Application No. 16/594,558 is a continuation-in-part of application No. 15/365,560, filed on Nov. 30, 2016, now abandoned.
(60) Provisional application No. 62/261,099, filed on Nov. 30, 2015.

(51) Int. Cl. A61B 5/00; A61C 7/08; A61B 90/00; G16H 15/00

(52) U.S. Cl. CPC A61B 5/4833; A61C 7/08; A61B 5/6802; A61B 5/682; A61B 90/361; G16H 15/00; A61B 5/7235; A61B 2090/364

(57) ABSTRACT

A system for preparing a prescription for the use of one or more orthodontal correction devices, such as aligners, which allows for remote interaction and guidance by dental professionals with their patients. The aligner may include a sensor to detect when it is being worn for tracking compliance and determining when the next correction device within the prescription should replace the current correction device. A software application associated with the patient allows them to view and track their progress and to send data to their dental professional, and similarly the dental professional is alerted by the present invention when compliance issues or other issues may arise requiring intervention.

### CROSS-REFERENCE TO RELATED APPLICATION

This application is a continuation-in-part of and claims priority in U.S. patent application Ser. No. 16/594,558, filed Oct. 7, 2019, which is a continuation-in-part of U.S. patent application Ser. No. 15/365,560, filed Nov. 30, 2016, which claims priority in U.S. Provisional Patent Application No. 62/261,099, filed Nov. 30, 2015, and also claims priority in U.S. Provisional Patent Application No. 62/934,208 filed Nov. 12, 2019, and is a continuation of U.S. patent application Ser. No. 17/095,933, filed Nov. 12, 2020, all of which are incorporated herein by reference.

### BACKGROUND OF THE INVENTION

### 1. Field of the Invention

The present invention relates generally to an orthodontal correction system and method for use thereof, and more specifically to computerized system for monitoring and adjusting an orthodontal correction system.

### 2. Description of the Related Art

Orthodontal correction for straightening crooked teeth date back thousands of years. Modern orthodontal correction devices include the standard metal braces and retainers. Newer devices are designed for subtle changes to teeth over time by swapping out wearable devices that can be worn at all times. Branded as Invisalign® and released in 1999, Align Technology of San Jose, Calif. offered orthodontists the first commercial aligner system to straighten teeth. With the proliferation of intra-oral scanners, which replace “messy” impressions, and 3D printing, some orthodontic clinics are even producing their own brand of in-house aligner systems.

Treating orthodontic patients with aligners is quite different than treating orthodontic patients with traditional braces. Because of this, aligners often cause some disruption to clinic workflow. While it is true that the advent of aligner therapy introduced some efficiencies, it also brought some unexpected inefficiencies. The splintering of the aligner market has further disrupted clinic workflow and added clutter, noise and confusion to the treatment process. Each aligner system requires a different user/doctor interface, separate database, etc. A significant amount of time is used simply to compile information needed to begin the aligner appointment. This leads to wasted chair time for the clinic. Chair time is what limits production and it should never be wasted.

Meanwhile, the aligner market continues to expand, and the aligner patient is being influenced by “do it yourself” aligner brands. Some aligner patients desire to perform the aligner treatment themselves and receive their aligners through the mail, without ever seeing a doctor. Other aligner patients want a doctor to closely monitor their aligner treatment in the office every 4-6 weeks. Most aligner patients, however, would like to have a combination of these two treatment methods. Today's aligner patient desires the convenience of minimal office visits that “direct to consumer” aligner brands offer, yet still have a doctor available to plan and monitor their treatment. Patients report that, should problems arise, they would like to have access to office appointments and face-to-face time with a doctor to fix or eliminate the problem and assure them that their aligner treatment is progressing as it should. The first problem is that today's aligner patient cannot currently have all they want; the patient must choose between traditional, in office, appointment-based treatment, or unsupervised “home alone” treatment.

Heretofore there has not been available a system or method for orthodontal correction monitoring with the advantages and features of the present invention.

### BRIEF SUMMARY OF THE INVENTION

The present invention generally provides supervision by tracking the patient's aligner treatment and providing both the doctor and the patient individualized real-time feedback via a software application. If the patient's behavior and feedback are good, and with the option for Virtual Visits through the app, the patient will require fewer (if any) visits at the office. In cases where the patient's compliance is poor or they are concerned about treatment, negative feedback is generated and the patient and doctor are alerted to communicate via messaging, a virtual visit, or to schedule an in-office appointment. The doctor is then able to make the needed adjustments and get the aligner treatment back on track and the patient's questions and concerns are addressed. All the communication features that are available on today's smart phones, such as text, email, video chat, etc. can be accessed through the app, with full HIPAA compliance, for real time two-way doctor-patient communication. This gives the at home aligner patient a “safety net” to confidently flow through aligner therapy reducing, and possibly eliminating, future office visits. The benefits of reducing or eliminating office visits to the patient and to the doctor cannot be overstated. In the event of prior data from patient self-reporting wearing of alignment devices or sensors reporting the wearing of alignment devices, the dental expert can determine that a reduced number of visits could be set-up at the initial meeting with the patient. This value can be altered further as the patient wears (or fails to wear) their device, based on future self-reporting and/or sensor reporting data.

The second problem is managing, and communicating with, individual aligner patients during an office or tele-health visit. It is an inefficient process. Time is wasted trying to determine which stage of aligner treatment the patient should currently be at (some patients could have more than 40 stages). It can be difficult to determine if the patient is ahead or behind their prescribed treatment stage and whether or not they are staying on schedule. The inefficiencies are compounded when the patient population is composed of patients being treated with various aligner brands. Overall, it can be very disorienting for an orthodontist seeing 80-120 patients a day.

The solution is the present invention's software dashboard feature on the doctor's console. All patients are listed on the dashboard and color-coded, so the doctor knows quickly, with a glance at the dashboard, whether the individual patient is on track, progressing smoothly and pleased with their treatment, or if they need assistance. Tapping on the individual patient icon brings up all of the patient's data, history and previous communications between the doctor and the patient. The app merges patients and their data from any/all aligner systems into a unified dashboard streamlining treatment of the aligner patient—whether the patient is in the office or being seen via the tele-health feature.

The system may also use artificial intelligence and/or machine learning to evaluate compliance and reported patient data in order to make recommendations so that eventually the system could make autonomous adjustments to treatment protocol, however until such advancements are made the machine learning determinations assist the doctor and patient during treatment. For instance, if the patient has been consistently wearing the aligners for “x” percent more hours than specified over the last “y” days, with appropriately positive feedback, the system would send an alert to the doctor suggesting an increase in aligner change frequency. Similarly, if the system detects inconsistent reporting or concerning data/compliance, it will notify and message the patient with suggestions on how to get back on track and improve their results.

The system can also help to track and monitor daily oral care through self-reporting by the patient. The patient may also perform regular self-reporting of the status of their corrective device by providing photographs of their current corrective device to compare with photographs of no corrective device and with the next corrective device to be worn. This information could lead their dental professional to alter the patient's schedule.

### BRIEF DESCRIPTION OF THE DRAWINGS

The drawings constitute a part of this specification and include exemplary embodiments of the present invention illustrating various objects and features thereof.

FIG. 1 is a three-dimensional isometric view of an orthodontal corrective device embodying elements of the present invention and diagramming their interaction with a personal computing device.

FIG. 2 is a diagram showing the communications relationship between computing device elements of an embodiment of the present invention.

FIG. 3 is a diagrammatic representation of an example user interface of a software application at a dental professional's computing device showing an overview dashboard.

FIG. 4 is a diagrammatic representation of an example user interface of a software application at a dental professional's computing device showing an image viewing dashboard.

FIG. 5 is a diagrammatic representation of an example user interface of a software application at a dental professional's computing device showing an image comparison function.

FIG. 6 is a diagrammatic representation of an example user interface of a software application at a dental professional's computing device showing a prescription generation dashboard.

FIG. 7 is a diagrammatic representation of an example user interface of a software application at a dental professional's computing device showing data analysis and calculations aimed at tracking progress of the present invention.

FIG. 8 is a diagrammatic representation of an example user interface of a software application at a patient's computing device showing data analysis and calculations aimed at tracking progress of the present invention.

FIG. 9 is a diagrammatic representation of an example user interface of a software application at a patient's computing device showing accessible features and functions available for exploring aspects of the present invention.

FIG. 10 is a diagrammatic representation of an example user interface of a software application at a patient's computing device showing self-report indicators associated with generating data used with the present invention.

FIG. 11 is a flowchart diagramming the steps practiced in an embodiment of the present invention.

FIG. 12 is a flowchart diagramming additional steps practiced in an embodiment of the present invention.

FIG. 13 is a continuation of the flowchart thereof.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art of how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Orthodontal Correction System 2

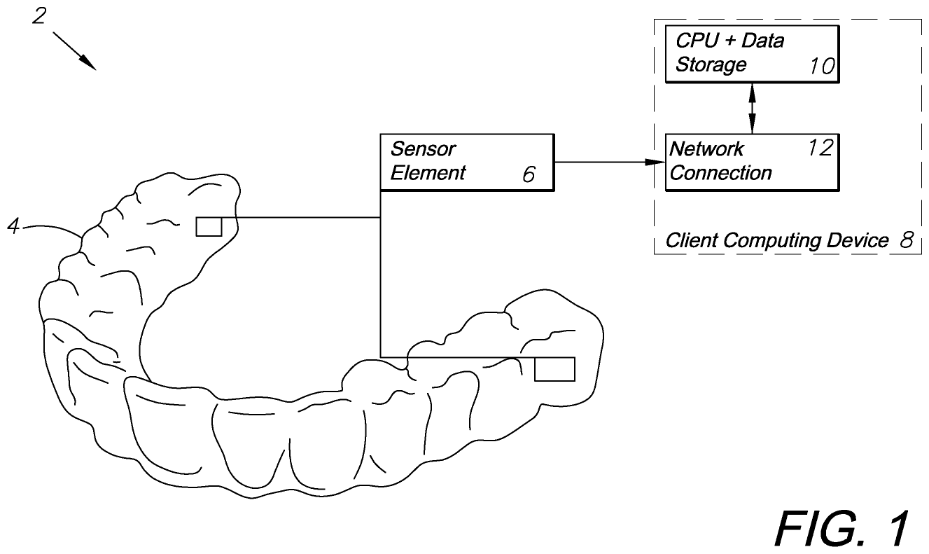

FIG. 1 shows an orthodontal correction system 2 where a wearable corrective device 4 includes sensors 6 for detecting when the corrective device 4, such as an orthodontal aligner, is being worn. These sensors could be temperature sensors, humidity sensors, or other sensors or combinations of sensors which can detect if the corrective device 4 is being worn in the mouth of the user. The sensor elements 6 communicate wirelessly with a patient computing device 8, such as a smartphone or laptop computer. Alternatively, these sensor devices could interact with a proprietary computing device or directly with a wireless communications network to deliver electronic communications when the corrective device 4 is being worn by the user. The computing device 8 should at least include a central processing unit (CPU) and data storage 10 as well as a network connection 12 for connecting to the wireless network. It should be noted that the entire system 2 could proceed only on client self-reported data of how often they are wearing their corrective device and no sensor within the corrective device is required.

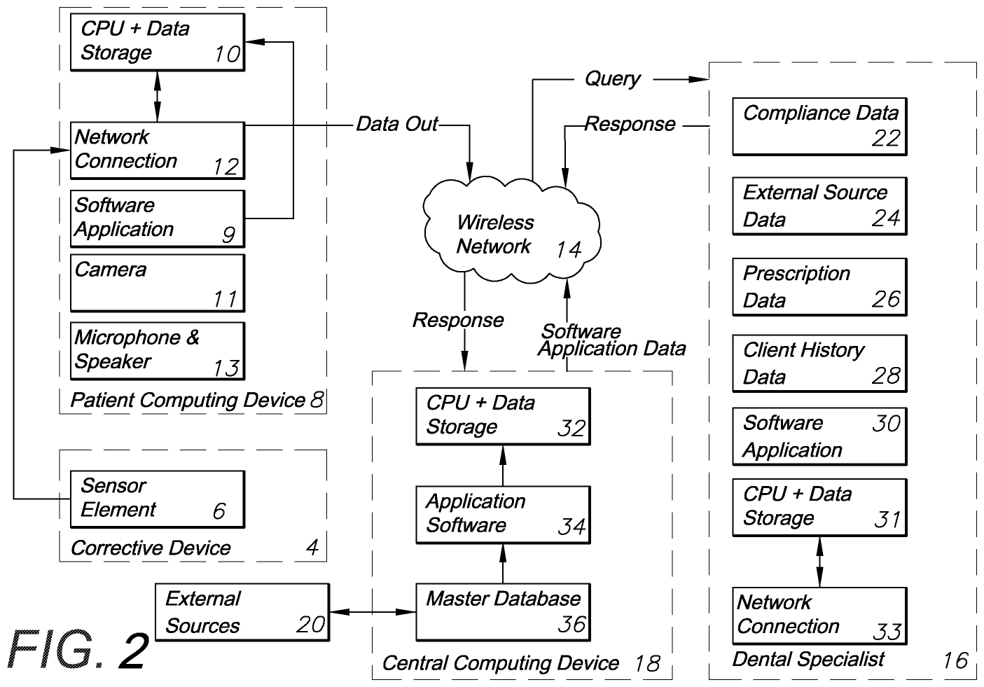

FIG. 2 shows the communications network where the patient computing device 8 communicates through the wireless network 14 with a remote central computing device 18 such as a central server which may house the software application 9, and a dental specialist 16 computing device. A software application 9 is stored on the patient's computing device 8 for accessing and interacting with the central computing device 18 and the dental specialist 16 via the wireless network 14. The patient's computing device 8 includes a camera 11 for taking pictures of the patient wearing their corrective device 4 for comparison and analysis by the dental specialist. A speaker and microphone 13 may also be necessary for having remote conversations with the dental specialist.

The dental specialist computing device allows the dentist or orthodontist to determine whether the patient is wearing the corrective device 4 according to a predetermined schedule to keep the corrective prescription on track. The dental specialist contains compliance data 22, such as a plan and historical patient data regarding their oral history, external source data 24 from external third-party sources 20 (e.g. third party manufacturers), prescription data 26 describing a plan for correcting the patient's teeth, patient history data 28 which may contain other history including past compliance, and a software application data 30 which may provide a graphical view of information relevant to the patient so the dental specialist can quickly ascertain what is going on. Like typical computing devices, the dental specialist computing device 16 also includes CPU and data storage 31 and a network connection 33 for connecting with the wireless network 14.

The central computing device 18 is the core analysis element of the present invention. While the dental specialist is fully capable of overriding any determinations made at the central computing device 18, this device can help the dental specialist made decisions based on progress measured by the device. The CPU and data storage 32 stores the application software 34 which can obtain data from the master database 36 which may include external data sources 20, patient data, and dental specialist generated data. External data sources 20 may include ideal dental models, health data, and other data that simply may not come from the patient or the dental specialist.

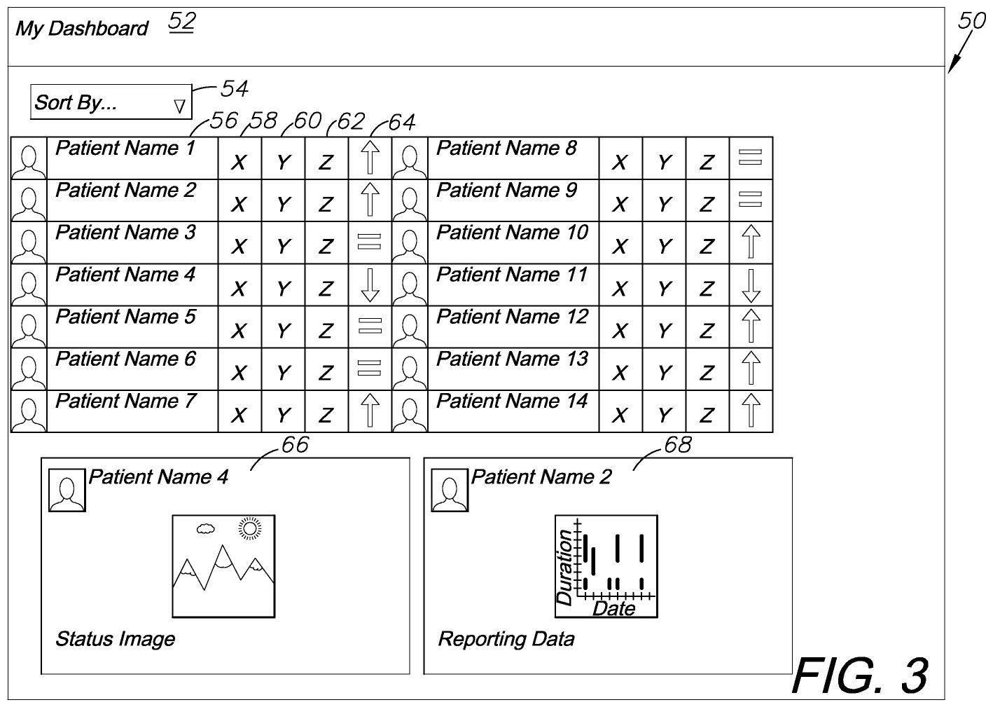

FIG. 3 shows an example of a user interface 50 that may be displayed by the dental specialist's computing device 16. This example user interface 50 shows the title 52 as “my dashboard,” Here, a list of the specialists' patients can be seen at a glance, along with basic information on each patient. Each patient has a patient photograph and patient name 56 along with a brief description of the patient's treatment goals. Three example data points are shown, such as a self-reported discomfort 58, if any, self-reported oral care status 60, and a 1-100% level of compliance 62 which is automatically calculated from the tracked wearing of the correction device 4 by the patient. Other data points could replace or be added to these, such as measured duration of wearing the correction device 4 on that day or the number of days remaining until the next correction device should be used. An indicator arrow 64 may also be included, showing trends of compliance either upward, downward, or staying the same (showing an equal sign). Beneath the list of patients could be messages, images, or other information that the specialist desires to see or which may have arrived from patients for the specialist on that day. For example, a message 66 showing a status image from “Patient Name 4” could show up or a message 68 with a status chart showing compliance of “Patient Name 2” might appear.

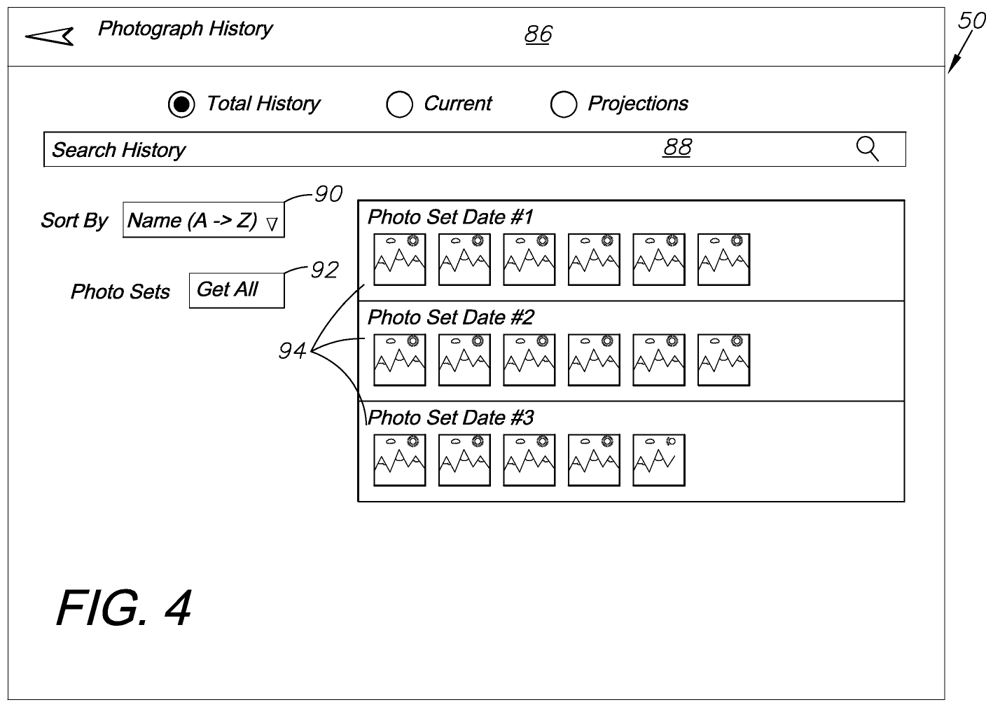

FIG. 4 shows the user interface 50 displaying a titled “photograph history” view 86. Here, the dental specialist can review past and current photographs of a selected patient's status of wearing a corrective device 4. Future projections may also be stored here as well, for example, if models have been generated. As shown here, the total history of a patient's images are viewable. The specialist can use a search history 88 bar to look at specific patient names, and the data can be sorted using a sort feature 90. Photo sets can then be retrieved using the “get all” button 92. Photo sets 94 may be separated by date or by type of photo, e.g. photos showing wearing current device, wearing no device, and wearing the next device in the prescription.

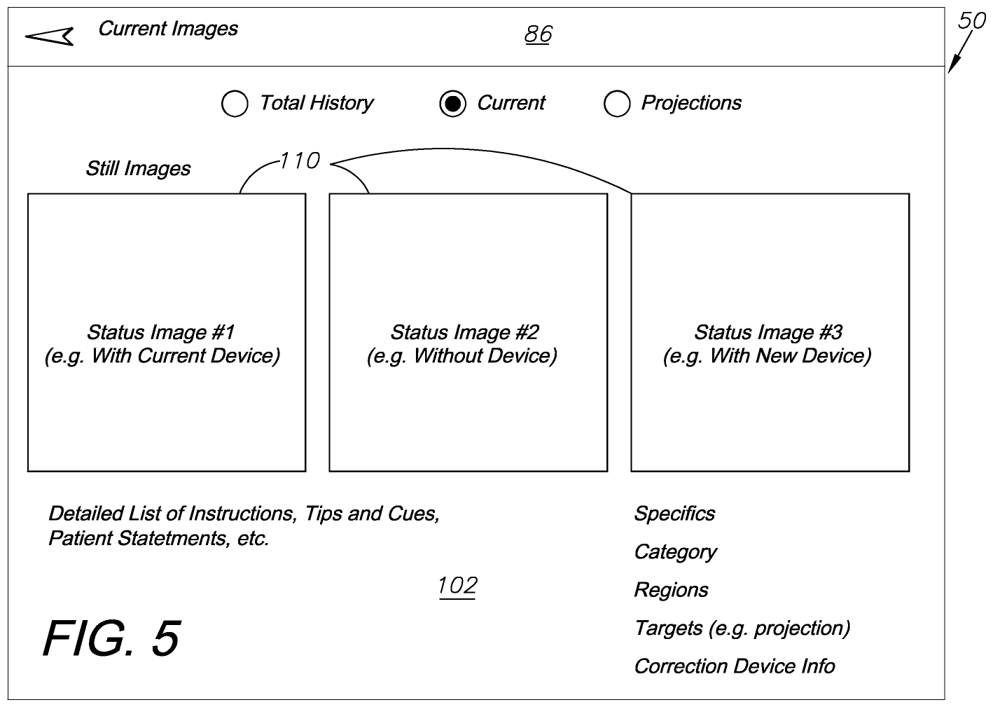

FIG. 5 shows the user interface 50 displaying the current images 86 view, with three still images 110 for a chosen patient. When changing to a new correction device, the patient takes a first photograph with their computing device 8 camera of their teeth with the old correction device, a second photograph with no correction device, and a third photograph with the new correction device being worn. This is sent to the dental specialist so that they can determine if there are any issues without an in-person visit. Other data 102 associated with the patient is also viewable below the images so that the dental specialist can quickly reference what the ultimate goals are.

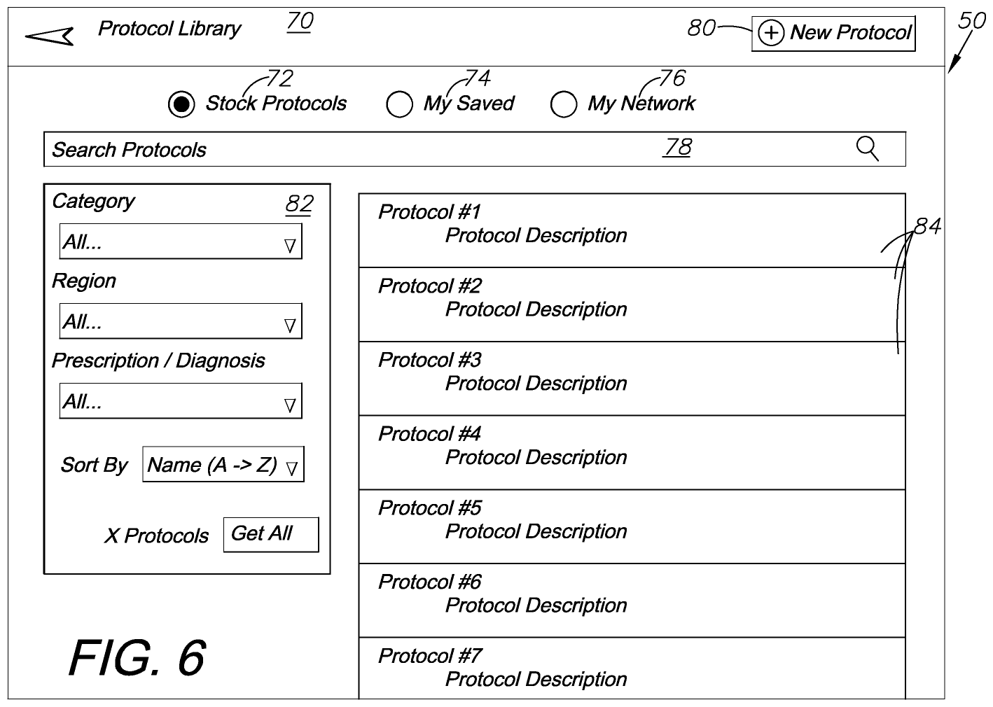

FIG. 6 shows the user interface 50 showing a protocol library 70 where the dental specialist can create new plans or update existing plans for patients. These protocols 84 may differ from corrective device to corrective device, may include oral health instructions, or other related instructions to be automatically delivered to the patient throughout the completion of their dental correction plan. New protocols can be created using the appropriate button 80. A number of stock protocols 72 may be provided from the central computing device for dental specialists to pick up and use while using the software application, and the dental specialist can also view protocols they've saved via the “my saved” 74 list or other protocols created or saved by other specialists in their network via the “my network” 76 list, which may include other specialists in their practice. They may also search all or narrowed protocols via the search bar 78. The protocols available to the specialist can be narrowed using the tools 82 to sort or find protocols to be prescribed to specific patients.

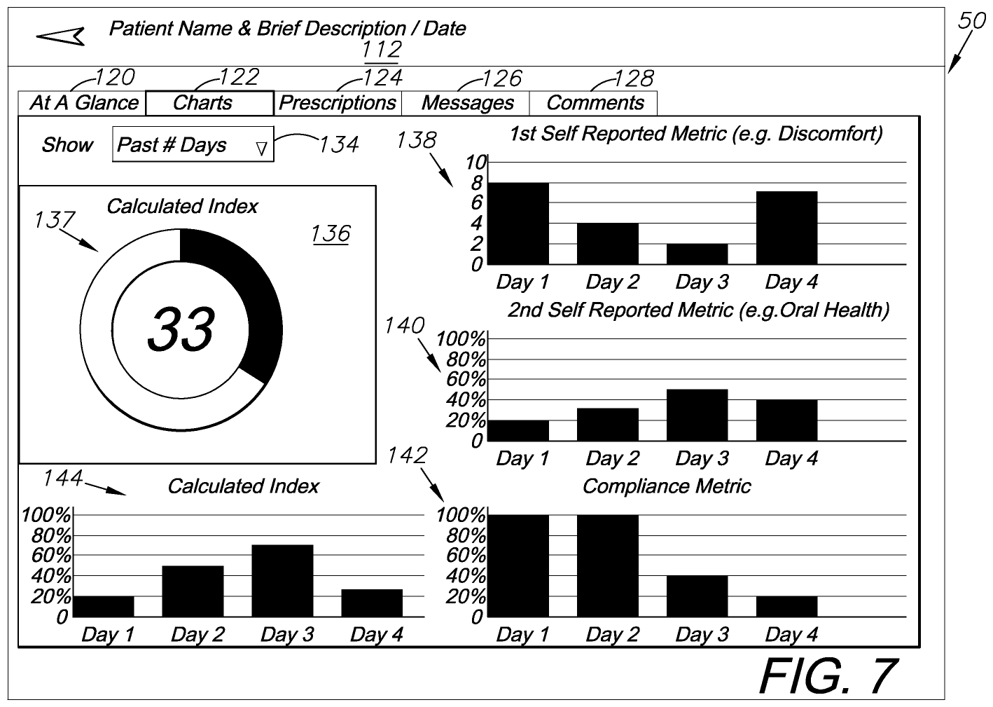

FIG. 7 is the user interface 50 showing the core of the software application 30 used by the dental specialist to make determinations about how well a patient, whose name appears at the top 112 of the tabs, is performing and whether to adjust that patient's protocols going forward. At the core of this is the calculated index 136 which is generated by including user-input data, data from the sensor 6 within the corrective device 4, and potentially data from the dental specialist upon reviewing photographs and other information provided by the patient to the specialist. It could even take into account computer-based comparisons of the photos sent in by the patient. This calculated index is a calculated measurement of the treatment results in an ongoing, updatable calculation. The index wheel 137 as shown could be color-coded so that as the number approaches 100% result the color would be green, whereas the closer to 0% it would be red.

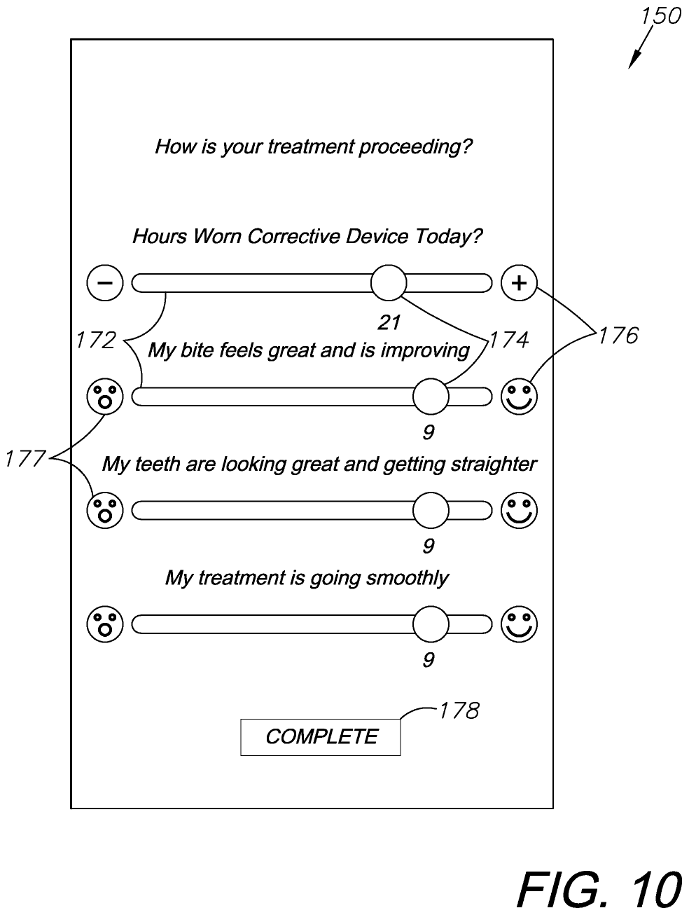

Data reported from the patient can include several different metrics 138, 140 as well as the compliance metric 142 from the sensor 6 to result in the calculated index 136 which can also be charted over days as shown in 144. FIG. 10 shows some of the metrics that the user may input. From this user interface 50 the specialist can change the number of days 134 that they are viewing for this patient, or can change from the “charts” tab 122, containing all of this information, to an “at a glance” tab 120 for a quick overview of the patient, the prescriptions tab 124 as shown in FIG. 6, a messages tab 126 for communicating with patients via text, email, or even video chat, and a comments tab 128 for them to take notes or generate other information.

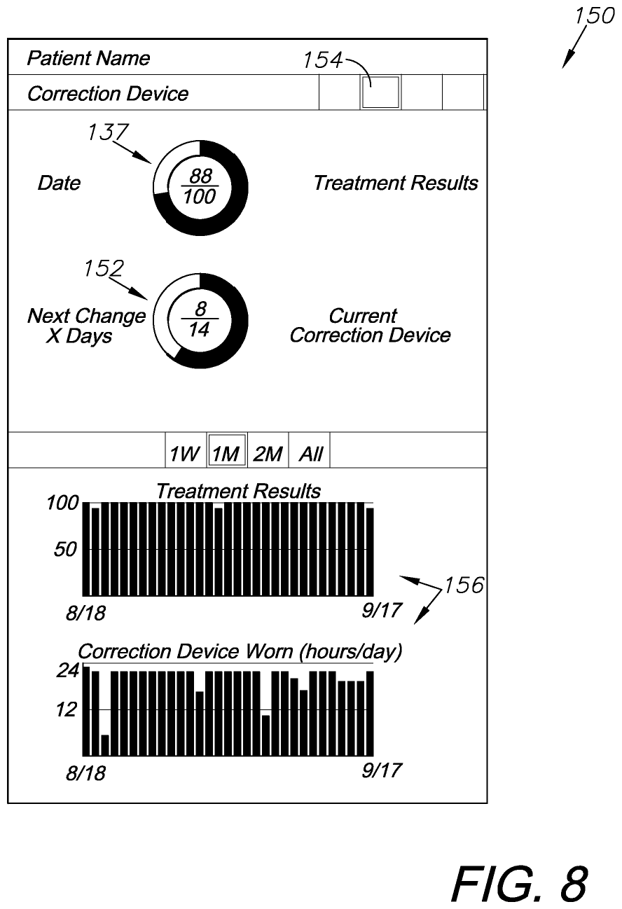

FIG. 8 shows a user interface 150 of the patient's computing device 8, with an active tab 154 showing the index wheel 137 with the calculated index, as well as a second wheel 152 showing where the patient is at in the overall prescription plan. Here, it is showing that the patient is currently on correction device #8 out of a total of 14. It can indicate when the next change is scheduled to occur assuming results continue to be good. Below these wheels are charts 156 which can show selectable time periods (e.g. 1 month) of the calculated index, hours the correction device is worn each day, and other relevant data points.

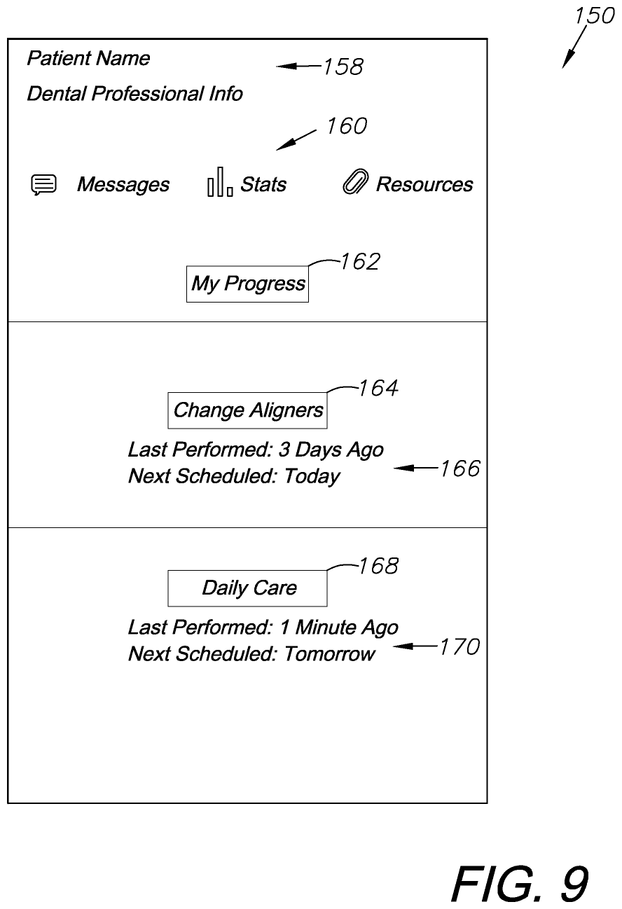

FIG. 9 shows the user interface 150 of the patient's computing device 8 with a different active tab which contains the patient name and dental professional information 158, selectable tabs 160 for viewing messages, statistics, resources, or other features of the software application 9 of the patient's computing device 8. Users can selectively view their orthodontal correction progress with a “my progress” button 162 which would take you to the information displayed on the user interface of FIG. 8. A status update 166 showing when corrective devices have been changed last and when the next scheduled change should occur appears below a selectable button “change aligners” 164 for when the patient is supposed to change their corrective device. When the patient selects this feature, the information 166 will update and a message will be sent to the central computing system 18 and the dental specialist 16, updating the respective databases and software applications located there. A similar status indicator 170 is associated with daily dental care (e.g. brushing and flossing), which indicates when the patient last pressed the “daily care” button 168 and indicating when the next scheduled time for daily care should occur.

FIG. 10 shows the user interface 150 of the patient's computing device 8 with another different active tab which shows several self-reporting interfaces 172, shown here as sliders having indicators 174 which can be adjusted up using a first selector 176 or down using a second selector 177. Assuming the patient's computing device 8 includes a touch screen interface, these indicators 174 could be moved using touch interface by sliding them along the sliders. Other self-reporting interfaces could also be used, such as simple numbers, up and down arrows, or blank text boxes for entering information. These self-reporting interfaces 172 allow the user to provide feedback that is not automatically detected using the sensor 6 of the correction device 4 or other data which can be interpolated automatically or by the dental professional (e.g. through pictures taken by the patient). Such data may include a self-report of how long they have worn their corrective device that day (in the event the sensor 6 is malfunctioning), how satisfied the patient is with the performance of their corrective device and prescription plan, and how comfortable the patient is. This data is fed into the entire analysis when completed 178 and may be included when calculating the calculated index 136.

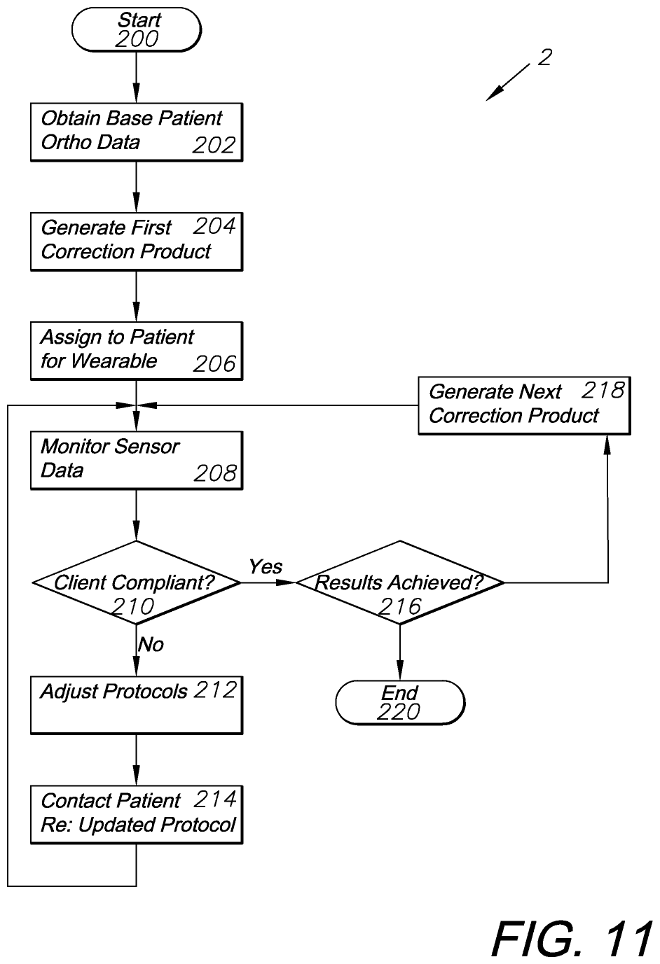

FIG. 11 steps through a high-level method of implementing the present invention. The process starts at 200. Base patient orthodontal data is obtained at 202 when the patient visits the specialist to determine a protocol for correcting the patient's teeth. A first correction product is generated at 204 and assigned to the patient at 206 along with a protocol for when to wear the device. Sensor data within the wearable device is constantly monitored at 208 and it is determined whether the patient is wearing the device according to the designed protocol. A check occurs whether the patient is compliant at 210, meaning whether the patient has worn the device an appropriate amount of time and for appropriate durations to achieve the goals of the protocols. If the patient has met or exceeded these goals, a second check at 216 determines if the results of the protocol have been achieved. If yes, the process ends 220 and the user may continue to wear the device as needed. If results haven't been achieved at 216, the next product is generated at 218 to move along the predetermined protocol, and the sensors of the new device are monitored at 208.

Alternatively, the sensor element may not be included in the alignment device and sensor-reporting data can be replaced with patient self-reporting data, similar to the self-reporting data used in U.S. patent application Ser. No. 15/365,560 (now U.S. Publication No. 2017/0154161) and Ser. No. 16/594,558, owned by the same entity as the present application, and which are incorporated herein by reference. This self-reporting system could be used in place of or in conjunction with the sensor-reporting features discussed above. Such a step would occur along with step 208 of FIG. 3 referenced above.

If the patient is not compliant at 210, protocols may be adjusted at 212, meaning the patient may have to wear the device for a longer period. The specialist will update the patient and contact them at 214, which will hopefully result in the patient wearing the device according to the protocol. Again, the sensors are monitored at 208, and the process continues until the patient results are achieved at 216.

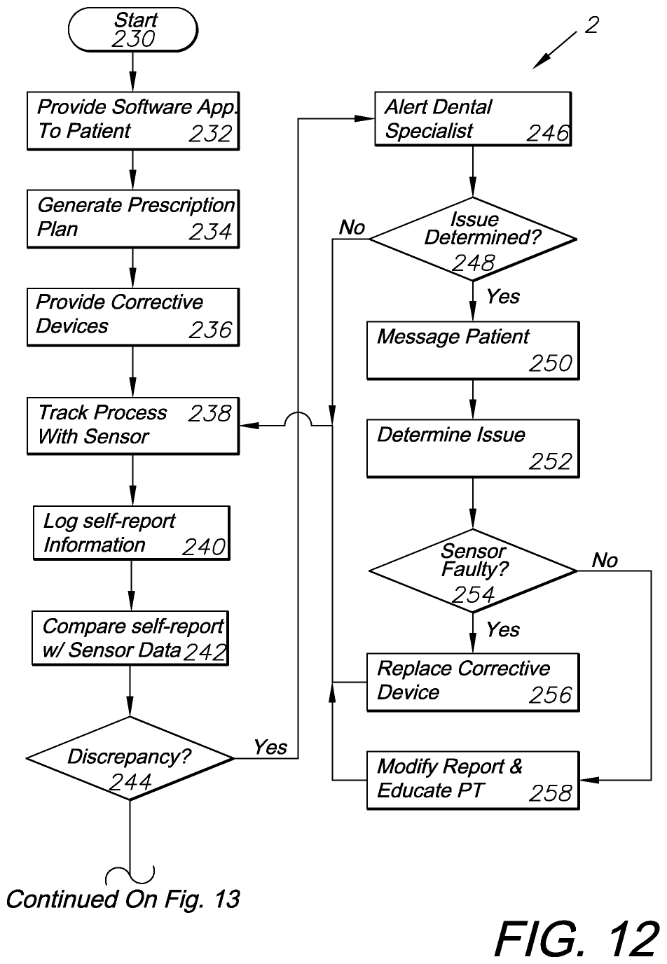

FIG. 12 shows the steps taken in automatically tracking and calculating the calculated index 136 in more detail, as well as how the patient, dental professional, and the software application and its associated data are used to proceed through the prescription and provide orthodontal correction with the correction devices 4. The process starts at 230 and the patient obtains the software application 9 on their computing device 8 at 232. A prescription plan is generated at 234 by the dental professional, which includes general daily care and one or more in a series of corrective devices used to provide orthodontal correction to the patient's teeth. The corrective devices 4 are provided to the patient at 236. The patient may be given one corrective device or may be given multiple corrective devices based upon the determined prescription. In the event of only a single device being given, this will require the patient to obtain additional corrective devices as the prescription progresses.

The system tracks how often the patient is wearing the corrective device 4 with its sensor 6 at 238, and also measures logged self-report information at 240, which includes information described in reference to FIG. 10 including self-reported time wearing the corrective device, pain or discomfort, and the patient's feelings regarding progress. The system will compare the self-reported data regarding how often they have worn their corrective device with the data being reported by the sensor at 242. A check of whether a discrepancy exists between these two data points occurs at 244. If a discrepancy is detected at 244, then the dental specialist is alerted at 246. The dental specialist checks the reported data, along with photos provided by the patient and other external and internal data, and determines whether there is an issue that needs to be explored at 248. If not, the process goes back to tracking with the sensor at 238 and the self-report data at 240.

If an issue is determined at 248, the dental specialist will message the patient at 250 and they will together determine the issue at 252. This may include having a video call with the patient, inspecting the corrective device, and other related steps. If the determination at 252 results in determining a faulty sensor 6 in the corrective device 4 at 254, then the corrective device is replaced at 256 and the process returns to step 238. If the sensor is not faulty, the dental specialist will update the self-report data to comply with the sensor data and will educate the patient on accurately reporting of when they are wearing the corrective device 258. The process then returns back to step 238.

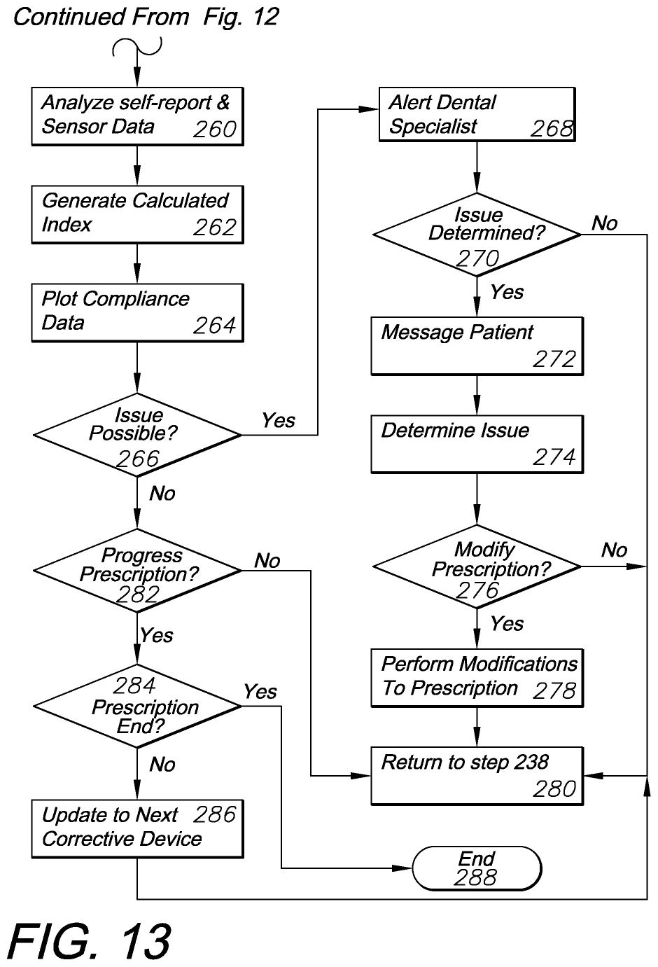

If no discrepancy exists at 244, the process continues on FIG. 13, where the system will analyze the self-report data and the sensor data at 260. This data is used to generate the calculated index at 262 which helps to show the patient and the dental specialist how well the prescription is proceeding. Compliance data is also plotted into charts and diagrams at 264 for easy reference by both the patient and the dental specialist. A determination is made at 266 by the system as to whether an issue might be possible. This might include an indication by the patient that they are in extreme discomfort or some other issue may be present, or it might be based upon the patient's failure in wearing their device. The issue at 266 could also be based upon data indicating a better-than-expected result of wearing the corrective device 4, which may end up in speeding up the prescription plan.

If a possible issue is detected at 266, the dental specialist is alerted at 268 to make a final determination as to whether the issue needs to be addressed at 270. If there is a determination that an issue needs to be addressed, the dental professional will message the patient at 272 and determine how to resolve the issue at 274. This could include addressing the patient's failure to properly follow daily care protocols or wearing their device to addressing the patient's pain level or discomfort. A determination as to whether the prescription needs to be modified is made at 276. This may include replacing the correction device 4, upgrading to a new correction device, or extending the duration of the current correction device based upon the issues detected, such as failure to adequately wear the device. This decision could also be made or at least suggested by the system 2 through data analysis. If there is a determination to modify the prescription at 276, the steps described above are taken at 278 and the process returns to step 238 at 280. Similarly, if no modification to the prescription is determine to be necessary at 276, the process returns to step 238 at 280.

If there are no detected issues at 266, a check is determined at 282 whether the prescription is to progress forward to the next corrective device. If not, the process returns to step 238 at 280. If the prescription is to progress at 282, a check is determined whether the prescription has reached its end at 284. If the prescription has reached its end, the process ends at 288. Otherwise the prescription moves to the next corrective device at 286 and the process returns to step 238 at 280. This process continues until the prescription determination at 284 ends the prescription, and the entire process ends at 288.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. An orthodontal correction device prescription, compliance, and communications system comprising:
a central server, a first computing device, and a second computing device, each including a respective central processing unit (CPU), data storage, and a connection to a computer network;
said first computing device associated with a dental specialist and including a first microphone, a first camera, a first graphical user interface (GUI), a dental specialist software application stored on said first computing device, and a connection to said computer network;
said second computing device associated with a patient and including a second microphone, a second camera, a second GUI, a patient software application stored on said second computing device, and a connection to said computer network;
at least one orthodontal correction device, said orthodontal correction device configured to correct said patient's teeth and generated based upon a prescription;
compliance feedback data generated with said second computing device and sent to said central server and to said first computing device;
reporting data generated with said second computing device, wherein said reporting data comprises a selected value amongst a predetermined range, said reporting data sent from said second computing device to said central server and said first computing device;
a calculated index value calculated from said central server from said compliance feedback data and said reporting data, said calculated index further adjusted based upon frequency of data received from said second computing device; and
wherein a determination of whether to replace said orthodontal correction device is made by said central server based at least partially upon said calculated index.

2. The system of claim 1, further comprising:
said calculated index value comprising a numeric value on a scale from zero to one hundred; and
said calculated index further comprising a color, said color dependent upon said numeric value.

3. The system of claim 1, further comprising:
a sensor within said corrective device, said sensor configured to detect when said corrective device is being worn;
said sensor configured to communicate with said second computing device to record sensor data associated with when said corrective device is being worn; and
said compliance feedback data at least partially including said sensor data.

4. The system of claim 1, further comprising:
a first image captured with said second computing device, said first image associated with a patient's oral structure wearing a first orthodontal correction device;
a second image captured with said second computing device, said second image associated with said patient's oral structure without wearing an orthodontal correction device;
a third image captured with said second computing device, said third image associated with said patient's oral structure wearing a second orthodontal correction device;
wherein said first and second orthodontal correction devices are included in said at least one orthodontal correction device;
wherein said second orthodontal correction device is intended to succeed said first orthodontal correction device as part of said prescription; and
wherein said first, second, and third images are delivered to said first computing device.

5. The system of claim 1, further comprising:
said compliance feedback data comprising an input corresponding with a duration said orthodontal correction device.

6. The system of claim 1, further comprising:
said compliance feedback data comprising an input corresponding with a satisfaction level corresponding with said orthodontal correction device.

7. A computer-implemented communication method of connecting a patient with a dental specialist, the method comprising the steps:
accessing a communication system with a first computing device associated with the dental specialist, wherein said first computing device comprises a central processing unit (CPU), data storage, a graphical user interface (GUI), a microphone, and a camera, and wherein said communication system comprises a central server including a CPU, data storage, and a connection to a computer network;
generating a prescription associated with the, said prescription comprising patient orthodontal data;
providing at least one orthodontal correction device, said at least one orthodontal correction device configured to correct said patient's teeth based upon said prescription;
sending a notification to a second computing device, said second computing device associated with said patient, and said notification associated with said prescription, wherein said second computing device comprises a CPU, data storage, a GUI, a microphone, and a camera;
accessing said prescription with said second computing device;
generating reporting data with said second computing device, said reporting data comprising a selected value amongst a predetermined range, and transmitting said performance data to said central server;
generating compliance feedback with said second computing device, said compliance feedback related to a duration said at least one orthodontal correction device is worn;
generating a calculated index value with said CPU of said central server, said calculated index derived from said compliance feedback data and said reporting data;
adjusting said calculated index value upon receiving additional compliance data and additional reporting data from said second computing device at said central server;
assigning a numeric value on a scale from zero to one hundred to said calculated index value;
accessing said calculated index value with said first computing device; and
adjusting said prescription based upon said calculated index value.

8. The method of claim 7, further comprising the steps:
wherein said at least one orthodontal correction device comprises a current orthodontal correction device and a new orthodontal correction device, said new orthodontal correction device being intended to succeed said current orthodontal correction device as indicated by said prescription;
capturing a first image with said second computing device of said patient's teeth with said current orthodontal correction device being worn;
capturing a second image with said second computing device of said patient's teeth with no orthodontal correction device;
capturing a third image with said second computing device of said patient's teeth with said new orthodontal correction device being worn; and
transmitting said first image, said second image, and said third image to said first computing device.

9. The method of claim 8, further comprising the step of adjusting said prescription based upon a comparison of said first image, said second image, and said third image.

10. The method of claim 7, further comprising the steps:
collecting, with a sensor within said at least one orthodontal correction device, said compliance data; and
sending, from said sensor to said second computing device, said compliance data via a wireless communication.
